(19) United States
(12) Patent Application Publication
(10) Pub. No.: US 2019/0240148 A1
(43) Pub. Date: Aug. 8, 2019

(54) LUBRICANT COMPOSITION AND METHOD FOR PREPARING THE COMPOSITION

(71) Applicant: Tarukino Holdings, Inc., Seattle, WA (US)

(72) Inventors: Rodger Scott Riefler, Monroe, WA (US); Howard Lee, Seattle, WA (US); Chelsea Cebara, Seattle, WA (US)

(21) Appl. No.: 16/269,506

(22) Filed: Feb. 6, 2019

Related U.S. Application Data
(60) Provisional application No. 62/627,694, filed on Feb. 7, 2018.

(51) Int. Cl. A61K 9/00; A61K 31/352; A61K 9/06

(52) U.S. Cl. CPC A61K 9/0034; A61K 9/06; A61K 31/352

(57) ABSTRACT

A method for preparing a lubricant composition is presented. The lubricant composition includes a THC emulsion, CBD emulsion, water, terpenes, and a thickening agent. The THC emulsion, CBD emulsion, water, cannabis terpenes, and thickening agent are combined as a lubricant.

### CROSS-REFERENCE TO RELATED APPLICATION

This non-provisional patent application claims priority under 35 U.S.C. § 119(e) to U.S. Provisional Patent application Ser. No. 62/627,694, filed Feb. 7, 2018, the disclosure of which is incorporated by reference.

### FIELD

This application relates in general to a personal use lubricant, and in particular to a lubricant composition and method for preparing the composition.

### BACKGROUND

Personal lubricants are commonly used for sexual pleasure and comfort. Specifically, lubricants can reduce friction during sexual intercourse and masturbation, as well as help reduce the risk of tearing and injury, by adding moisture to an application area. Lubricants can be oil-, water-, or silicone-based and are generally available in a gel, liquid, or cream form. Oil-based lubricants can cause condoms to break and thus, should not be used with latex or polyisoprene condoms. However, water- and silicone-based lubricants are safe to use with all types of condoms.

Additionally, lubricants can have different textures, sensations, and flavors to increase pleasure experienced by a user during sexual intercourse or masturbation. For example, some lubricants include flavors, such as fruit flavors, while other lubricants include warming properties to warm a body region to which the lubricant is applied. However, conventional lubricants fail to increase blood flow, thereby resulting in longer and stronger orgasms during sexual intercourse or masturbation.

Accordingly, what is needed is a lubricant that can increase blood flow and provide prolonged and increased stimulation to a user during sexual intercourse and masturbation. Preferably, use of the lubricant can result in longer and stronger orgasms.

### SUMMARY

A personal lubricant that provides increased stimulation, which can result in stronger orgasms, can include THC or CBD, alone or in combination. For example, a lubricant composition can include a THC emulsion, a CBD emulsion, or a combination of the THC and CBD emulsions. Additionally, the lubricant composition can include a combination of the THC and CBD emulsions, and additional components, including a thickening agent, and a sweetener substance. Also, the lubricant composition can optionally include a Diluent Mix to dilute the compositions and change a pH of the composition. The personal lubricant can be used safely with all types of condoms and sex toys, and does not leave a stain on clothes or sheets upon contact.

In one embodiment, the lubricant composition can include a THC emulsion, a CBD emulsion, water, terpenes, and a thickening agent. Additionally, the lubricant can include agents that sooth and protect skin, such as Allantoin and Aloe Vera, as well as sweetener substances, celluostics, preservatives, and acidity regulators. The lubricant can have the form of a cream or lotion and can be applied to erogenous zones on a user's body or on devices to be applied to the user's body, such as sex toys.

A further embodiment provides a personal lubricant. The lubricant composition includes a THC emulsion, CBD emulsion, water, terpenes, and a thickening agent. The THC emulsion, CBD emulsion, water, cannabis terpenes, and thickening agent are combined as a lubricant.

### BRIEF DESCRIPTION OF THE DRAWINGS

FIG. 1 is a flow diagram showing, by way of example, a method for preparing a lubricant composition based on a THC emulsion.

FIG. 2 is a flow diagram showing, by way of example, a method for preparing a lubricant composition based on a CBD emulsion.

FIG. 3 is a flow diagram showing, by way of example, a method for preparing a lubricant composition based on the THC emulsion of FIG. 1 and the CBD emulsion of FIG. 2.

FIG. 4 is a flow diagram showing, by way of example, a method for preparing a diluent mix.

FIG. 5 is a flow diagram showing, by way of example, a method for preparing a lubricant based on the THC emulsion of FIG. 1, the CBD emulsion of FIG. 2, and the diluent mix of FIG. 4.

### DETAILED DESCRIPTION

Lubricants are often used during sexual intercourse or masturbation to increase pleasure by reducing friction and preventing tearing due to the frication. Some lubricants can include flavor or provide additional sensations, such as warming of the user's skin. However, a personal lubricant composition that includes Tetrahydrocannabinol (“THC”) and Cannabidiol (“CBD”), alone or in combination, can increase a user's sensation and pleasure, such as by increasing sensation, such as a tingling effect, and increasing orgasmic potential, including longer and stronger orgasms. Generally, cannabinoids are generally associated with dilating blood vessels, which can cause increased blood flow. Applying the lubricant, with the THC or CBD, to the skin of a user can have similar effects of increased blood flow at the area of application, such as an erogenous zone, including genitals.

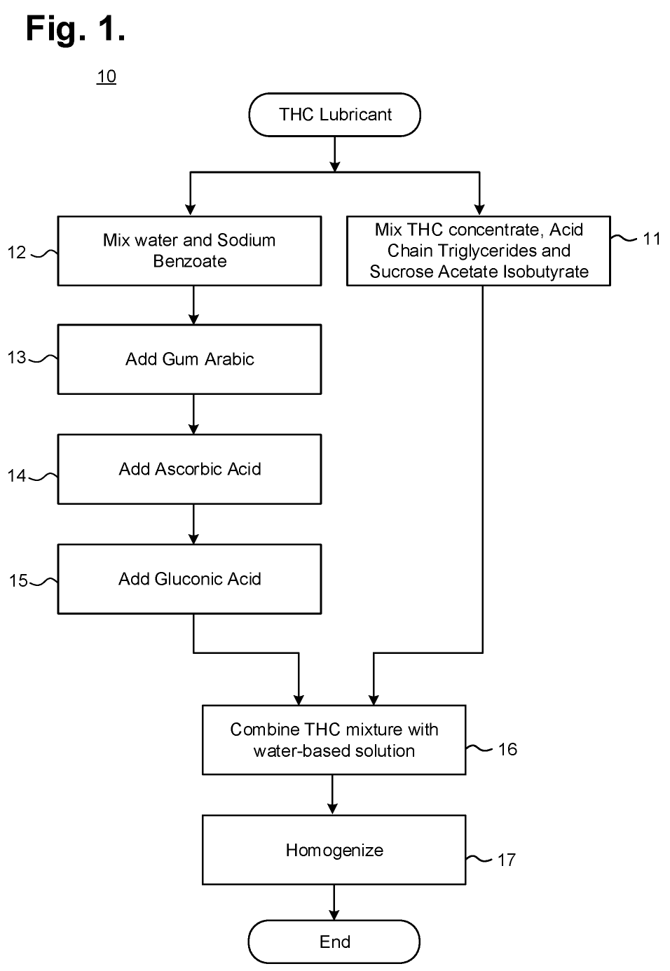

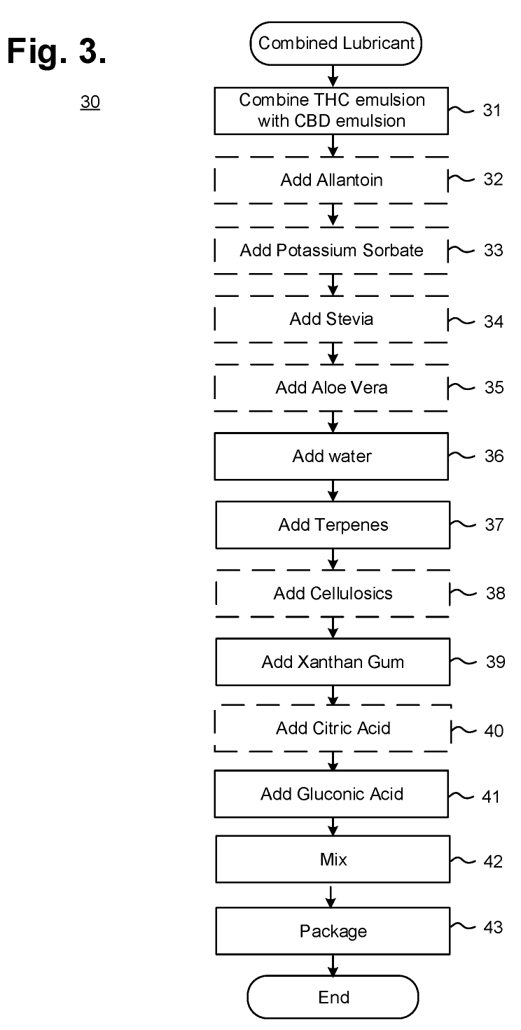

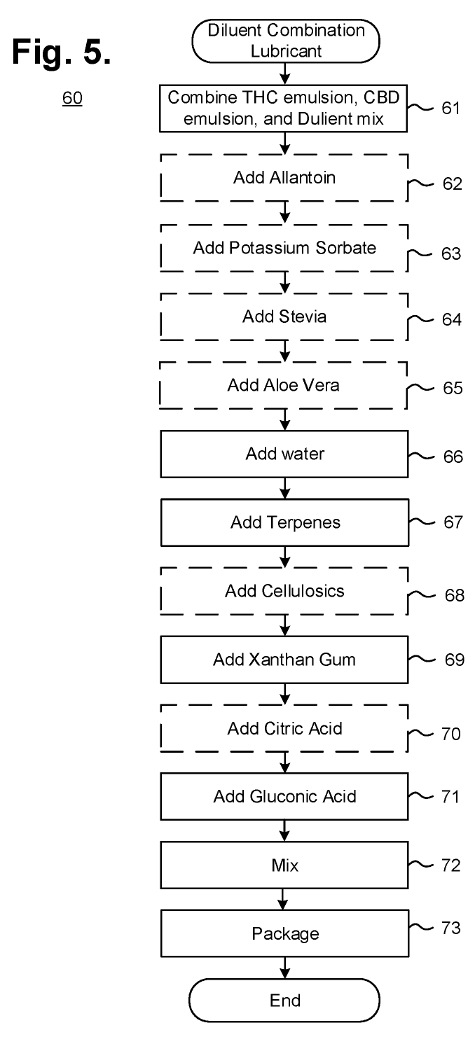

Increased blood flow can increase orgasmic potential, such as by prolonged or more intense orgasms. FIG. 1 is a flow diagram showing, by way of example, a method 10 for preparing a lubricant composition based on a THC emulsion. The THC emulsion can act as the lubricant composition by itself or in combination with other components, as further described below with respect to FIGS. 3 and 5. The THC emulsion can include the following:

TABLE 1
THC Emulsion

| | Range | Preferred Range |
|---|---|---|
| THC Emulsion | (percentage by weight) | (percentage by weight) |
| Water | 3-90% | 65-75% |
| Sodium Benzoate | 0.001-1% | 0.05-0.125% |
| Gum Arabic | 0.5 to 50% | 6-18% |
| Ascorbic Acid | 0.001-1% | 0.05-0.125% |
| Citric Acid | 0-3% | 0% |
| Gluconic Acid | 0-5% | 1.5-2.25% |
| THC concentrate | 0-30% | 2.5-4% |
| Mid Chain Triglycerides | 0-30% | 5-7% |
| Sucrose acetate | 0-30% | 0.75-1.75% |
| isobutyrate/Mid-chain | | |
| triglyceride (“MCT”) | | |

The THC emulsion can be prepared, for example, by mixing a water-based solution with and oil-based mixture. In one embodiment, the emulsion can be performed according to the method described in commonly owned U.S. patent application Ser. No. 16/010,082, filed on Jun. 15, 2018, pending, the disclosure of which is incorporated by reference, and as further described below. Specifically, the water-based solution can be prepared by adding deionized or purified water to a clean vessel and mixing the water with a high shear mixer at around 5,000 rpm. Other types of mixers and mixing speeds are possible. Sodium Benzoate, which acts as a preservative, can be added to the water, and mixing (step 12) of the water and Sodium Benzoate can continue until the Sodium Benzoate is completely dissolved. In one embodiment, the mixing continues for approximately 5 minutes to dissolve the Sodium Benzoate. However, other times are possible.

Subsequently, the mixing speed of the high shear mixer can be increased to approximately 8,000 rpm and Gum Arabic, which acts as a thickening agent or emulsifier, can be slowly added (step 13) to the water and Sodium Benzoate to avoid lump formation. However, other mixing speeds are possible. After all the lumps are removed, mixing can continue via the high shear mixer for around 10 minutes; however, other mixing times are possible. After mixing, the combination of water, Sodium Benzoate, and Gum Arabic has a liquid consistency.

The water-based solution also includes Ascorbic Acid, which is slowly added (step 14) to the water, Sodium Benzoate, and Gum Arabic mixture under agitation and can be mixed for around 5 minutes. The Ascorbic Acid can act as a natural preservative to prevent microbial growth, while preventing spoilage and preserving freshness. Optionally, Citric Acid can be added to the water, Sodium Benzoate, Gum Arabic, and Ascorbic Acid to aid in preservation or add a sour flavor. Under agitation, Gluconic Acid can be slowly added (step 15) to the water, Sodium Benzoate, Gum Arabic, and Ascorbic Acid, as the water-based solution, for approximately 5 minutes. Upon completion of the mixing time, the water-based solution can rest for approximately 12 hours at 38° F. to 80° F. After the rest time, the solution can be stored for longer periods at around 38° F.

Prior to, during, or after the mixture is at rest, an oil-based mixture is prepared (step 11). The oil-based mixture can include one or more of mid-chain triglycerides, sucrose acetate isobutyrate/MCT, and THC concentrate, which are mixed (step 11) using mild heat, of around 75° to 160° F., until the mixture is clear with no light refraction. The heating temperature can be dependent on a nature of the extraction, the broad spectrum through isolate, and an amount of high vapor pressure components, such as terpenes and a viscosity of the terpenes.

Subsequently, the oil-based mixture is set aside for later use. In a further embodiment, different combinations of the mid-chain triglycerides, sucrose acetate isobutyrate/MCT, and THC concentrate can be prepared as the oil-based mixture. The THC concentrate can be extracted from any cannabis plant variety, including marijuana, hemp, or synthetic plants, as well as other plants that include THC. The THC can be provided in any form, including having a full extraction profile or a broad spectrum profile, or as high purity distillates. THC having a full extraction profile can be extracted from the cannabis plant using water and can include wax and chlorophyll, whereas THC with a broad spectrum profile differs by the removal of the wax and chlorophyll. Additionally, a full or broad spectrum THC extract will also or can also include CBD, Cannabinol (“CBN”), and other cannabinoids. THC distillate also removes the terpenes and a majority of low boiling or volatile compounds from the cannabis plant.

Once prepared, the water-based solution and oil-based mixture is combined (step 16), as the THC emulsion. However, prior to combination, the water-based solution should come to room temperature or up to 180°. In one embodiment, the water-based solution is heated to 100° to 120° F. Once the temperature has been reached, the water-based solution can be mixed around 5,000 to 10,000 rpm. The oil-based mixture can be heated to a temperature between 75° and 160° F. depending on a nature of the THC extraction, such as the broad spectrum through isolate, and an amount of high vapor pressure components, such as the terpenes and a viscosity of the terpenes. For example, the lower the viscosity and higher terpene content, the lower the temperature required for the oil-based mixture. Once heated, the oil-based mixture can be added to the water-based solution via a syringe, such as at a location at the bottom of the high shear mixer mixing head. Other mixing speeds and heating temperatures are possible. The combined water-based solution and oil-based mixture are mixed for approximately 15 minutes at around 5,000 to 10,000 rpm, after the final syringe of THC mixture is added. However, other mixing times and speeds are possible. Subsequently, the mixed water-based solution and oil-based mixture are homogenized (block 17) between 500 psi to 20,000 psi using one or two passes through the homogenization process via a single stage homogenizer or a multi-stage homogenizer. If a multi-stage homogenizer is used, the pressure of the second pass can be 5-25% of the pressure used during the first pass. For example, a two stage homogenizer can be used for two passes of the homogenization process at a pressure between 3000-4500/300-800 psi. After the homogenization process is complete, the THC emulsion can be stored at room temperature, such as in dark glass bottles.

Other components for use in the THC emulsion are possible. For example, in place of water, water and a solvent combination can be used, while Potassium Sorbate and other preservative systems can be used for Sodium Benzoate. In lieu of or in addition to Gum Arabic, other water-based, high hydrophilic-lipophilic balance (“HLB”) emulsifiers, such as Q-Natural or Habo MonoEster P90 (Sucrose Monopalmitate) can be used. Additionally other emulsifiers may also be used, including modified food starches, sucrose fatty acid esters, modified vitamin E and oil soluble emulsifiers, such as polysorbates. Citric acid can be used in lieu of or in addition to Gluconic Acid and food grade oils with densities above 1.0, such as glycerol ester of wood rosin (ester gum) can be used in lieu of Sucrose acetate isobutyrate/MCT. Additionally, food grade oils, such as vegetable oil, can be used in lieu of the Mid-Chain Triglycerides.

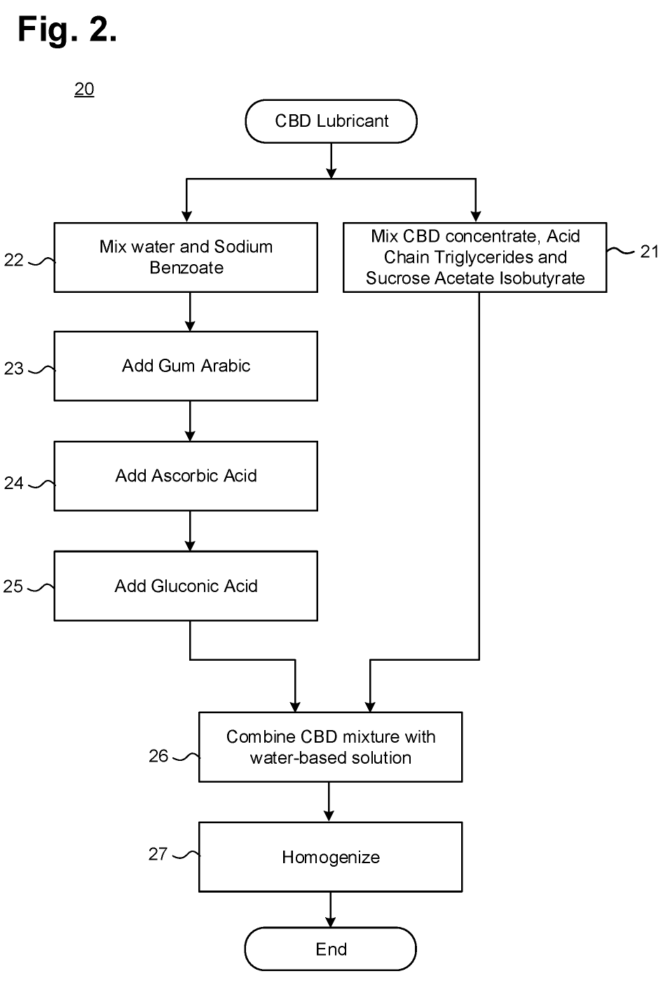

Alternatively, the lubricant can be prepared as a CBD emulsion, as described below, or can include the CBD emulsion as one of the components of the lubricant as described in detail below with respect to FIGS. 3 and 5. FIG. 2 is a flow diagram showing, by way of example, a method 20 for preparing a lubricant composition based on a CBD emulsion. The CBD emulsion includes the following components:

TABLE 2
CBD Master Mix

| | Range | Preferred Range |
|---|---|---|
| CBD Emulsion | (percentage by weight) | (percentage by weight) |
| Water | 3-90% | 65-75% |
| Sodium Benzoate | 0.001-1% | 0.05-0.125% |
| Gum Arabic | 0.5 -50% | 6-18% |
| Ascorbic Acid | 0.001-1% | 0.05-0.125% |
| Citric Acid | 0-3% | 0% |
| Gluconic Acid | 0-5% | 1.5-2.25% |
| CDB concentrate | 0-30% | 2.5-3.5% |
| Mid Chain Triglycerides | 0-30% | 5-7% |
| Sucrose Acetate | 0-30% | 1-1.75% |
| Isobutyrate/MCT | | |

The CBD emulsion can be prepared by mixing a water-based solution with an oil-based mixture. In one embodiment, the emulsion can be performed according to the method described in commonly owned U.S. patent application Ser. No. 16/010,082, filed on Jun. 15, 2018, pending, the disclosure of which is incorporated by reference, and as further described below. Specifically, the water-based solution can be prepared by adding deionized or purified water to a clean vessel and mixing the water with a high shear mixer around 5,000 rpm. Other types of mixers and mixing speeds are possible. Sodium Benzoate, which acts as a preservative, can be added to the water during mixing (step 22), which can continue until the Sodium Benzoate is completely dissolved, after approximately 5 minutes of mixing. However, other dissolution times are possible.

Subsequently, the mixing speed of the high shear mixer can be increased to approximately 8,000 rpm, and Gum Arabic, which acts as a thickening agent and emulsifier, can be slowly added (step 23) to the water and Sodium Benzoate to avoid lump formation. Other missing speeds are possible. After all the lumps are removed, mixing can continue via the high shear mixer for around 10 minutes or another mixing time. After mixing, the combination of water, Sodium Benzoate, and Gum Arabic has a liquid consistency.

The water-based acid can also include Ascorbic Acid, which is slowly added (step 24) to the water, Sodium Benzoate, and Gum Arabic mixture under agitation, and the mixture can be mixed for around 5 minutes or a different time. The Ascorbic Acid can act as a natural preservative to prevent microbial growth, while preventing spoilage and preserving freshness. Optionally, Citric Acid can be added to the water, Sodium Benzoate, Gum Arabic, and Ascorbic Acid mixture to aid in preservation or add a sour flavor. Further, under agitation, Gluconic Acid can be slowly added (step 25) over a time of about 5 minutes to the water, Sodium Benzoate, Gum Arabic, and Ascorbic Acid, as the water-based solution. Upon completion of the mixing time, the water-based solution can rest for approximately 12 hours at 38° F. to 80° F. After the rest time, the solution can be stored for longer periods of time, at around 38° F.

Prior to, during, or after the mixture is at rest, the oil-based mixture is prepared (step 21) and can include mid chain triglycerides, sucrose isobutryrate/MCT, and CBD isolate, which are mixed (step 21) using a mild heat of between 75° and 160° F. dependent on a nature of the CBD isolate, the broad spectrum through isolate) and an amount of high vapor pressure components, such as the terpenes and the terpene viscosity. For example, the lower the viscosity and higher the terpene content, the lower the temperature required to heat the oil-based mixture. The oil-based mixture should be heated until the mixture is clear with no light refraction. In a further embodiment, different combinations of one or more of the mid-chain triglycerides, sucrose isobutryrate/MCT, and CBD isolate can be used as the oil-based mixture. Subsequently, the oil-based mixture is set aside for later use.

The CBD isolate can be extracted from any cannabis plant variety, including marijuana, hemp, or synthetic plants, as well as other plants that include CBD. The CBD can be provided in any form, including having a full extraction profile or a broad spectrum profile, or as high purity distillates. CBD having a full extraction profile can be extracted from the cannabis plant using water and can include wax and chlorophyll, whereas CBD with a broad spectrum profile differs by the removal of the wax and chlorophyll. Additionally, each of a full or broad spectrum CBD extract can include THC, CBD, and other cannabinoids.

CBD distillate also removes the terpenes and a majority of low boiling or volatile compounds from the cannabis plant.

Once prepared, the water-based solution and oil-based mixture can be combined (step 26) as the CBD emulsion. However, prior to combination, the water-based solution should come to room temperature or should be heated to a temperature up to 180° F. IN one embodiment, the water-based solution is heated to a temperature between 100° and 120° F. Once at room temperature, the water-based solution can be mixed at around 5,000 to 10,000 rpm. The oil-based mixture, which can be heated to a temperature between 75° and 160° F. can be added to the water-based solution via a syringe positioned at the bottom of a high shear mixer mixing head. Other mixing speeds and heating temperatures can be used. The combined water-based solution and oil-based mixture are mixed for approximately 15 minutes at around 5,000 to 10,000 rpm, after all the oil-based mixture has been added. However, other mixing speeds and times are possible. Subsequently, the mixed water-based solution and oil-based mixture can be homogenized (step 27) between 500 psi to 20,000 psi using one or two passes through the homogenization process via a single stage homogenizer or a multi-stage homogenizer. If a multi-stage homogenizer is used, the pressure of the second pass can be 5-25% of the pressure used during the first pass. For example, a two stage homogenizer can be used for two passes of the homogenization process at a pressure between 3000-4500 psi for a first pass and 300-800 psi for a second pass. After the homogenization process is complete the CBD emulsion can be stored at room temperature, such as in dark glass bottles.

Other components for use in the CBD emulsion are possible. For example, in place of water, water and a solvent combination can be used, while Potassium Sorbate and other preservative systems can be used for Sodium Benzoate. In lieu of or in addition to Gum Arabic, other water-based, high hydrophilic-lipophilic balance (“HLB”) emulsifiers, such as Q-Natural or Habo MonoEster P90 (Sucrose Monopalmitate) can be used. Additionally other emulsifiers may also be used, including modified food starches, sucrose fatty acid esters, modified vitamin E and oil soluble emulsifiers, such as polysorbates. Citric acid can be used in lieu of or in addition to Gluconic Acid and food grade oils with densities above 1.0, such as glycerol ester of wood rosin (ester gum) can be used in lieu of Sucrose acetate isobutyrate/MCT. Additionally, food grade oils, such as vegetable oil, can be used in lieu of the Mid-Chain Triglycerides. In a further embodiment, the lubricant composition can include a mix of the THC emulsion and the CBD emulsion. FIG. 3 is a flow diagram showing, by way of example, a method 30 for preparing a lubricant composition based on the THC emulsion of FIG. 1 and the CBD emulsion of FIG. 2. The lubricant composition can include the following components:

TABLE 3
Lubricant Composition 1

| | Range | Preferred Range |
|---|---|---|
| Lubricant Composition 1 | (percentage by weight) | (percentage by weight) |
| THC Emulsion | 10-99% | 20-30% |
| CBD Emulsion | 5-99% | 7-10% |
| Allantoin | 0-1% | 0.125-0.2% |
| Potassium Sorbate | 0-1% | 0.0075-0.015% |
| Stevia | 0-2% | 0.125-0.15% |
| Aloe Vera | 0-5% | 0.125-0.15% |
| Water | 0-80% | 55-70% |
| Cannabis Terpene | 0-10% | 0.025-0.05% |
| Hydroxyethyl Cellulose | 0-5% | 0-0.1% |
| Xanthan Gum | 0-5% | 1-2% |
| Citric Acid | 0-3% | 0% |
| Gluconic Acid | 0-5% | 1.5-2.25% |

The lubricant composition can be prepared by combining (step 31) appropriate amounts of the THC emulsion and the CBD emulsion to a large mixing container. When the THC and CBD emulsions are prepared separately, THC and CBD assay values can be determined. The assay values can be used to ensure that final product ratios are met after combining the emulsions. However, a single emulsion with both THC and CBD can be prepared based on the steps described above with respect to FIGS. 1 and 2, and then used to prepare the lubricant, according to the steps listed below.

A large hole screen is positioned on a high shear mixer and the combined emulsions are mixed at approximately 6,000 rpm. The holes in the screen can be sized approximately ¼″, but can also be bigger or smaller. Optionally Allantoin, which is a skin conditioning agent, can be added (step 32) to the emulsion mix and mixed until dispersed. Dispersion of the Allantoin can take approximately 5 minutes of mixing. Potassium Sorbate, which is a preservative, can also be optionally added (step 33) and mixed until dispersed, which can take approximately 5 minutes. Further, a sweetener, such as Stevia, can optionally be added (step 34) and mixed, approximately 5 minutes, until dispersed. Also, a moisturizer, such as Aloe Vera, can be added (step 35) and mixed, approximately 5 minutes, until dispersed.

The addition (step 36) of water, such as deionized or purified water is optional and may be used when the THC and CBD concentrations require ratio adjustment. Terpenes, such as from cannabis plants, as well as other plants, can be added (step 37) and mixed until dispersed, such as for approximately 5 minutes. In one embodiment, the cannabis terpene can include Dutch Treat; however, other terpenes can also be used, including terpenes derived from non-cannabis sources. A cellulosic, such as Hydroxyethyl Cellulose, can be optionally added (step 38) and mix at an increased speed of approximately 9,000 to 10,000 rpm, while gently stirring with a spatula to ensure all lumps are dispersed. Mixing of the Hydroxyethyl Cellulose with the rest of the mixture described above can occur for approximately 10 minutes. However, other times are possible, as well as other cellulosics, such as Carboxymethyl Cellulose.

A thickening agent, such as Xanthan Gum, can also be added (step 39) to the mixture at the increased speed of approximately 3,000 to 10,000 rpm, while gently stirring with the spatula to ensure all lumps are dispersed. Citric Acid, which aids in preservation, can be optionally added (step 40) to the mixture and Gluconic Acid, which is commonly used as an acidity regulator, can be added (step 41) to the mixture. Once all the desired components have been added, the mixture is mixed (step 42) for approximately 10 minutes and then packaged (step 43), such as in bottles or jars.

Components, other than those described above, are also possible for use in the lubricant composition. For example, in lieu of or in addition to Allantoin, Aloe Vera, palm oil, vegetable oil, or glycerin can be used, as well as other healing or soothing components. Additionally, in lieu of or in addition to Stevia, sweeteners, such as Sucralose, Aspartame, cyclamates, Saccharin, Aceslfame Potassium, and Neotame can be used. In lieu of or in addition to Xanthan Gum, other food grade water soluble thickeners are possible, such as hydrocolloids, minerals such as fumed silica, and starches, as well as other thickeners. Other components that can be used include Sodium Benzoate, such as a replacement or in addition to the Potassium Sorbate. Finally, other terpenes are possible.

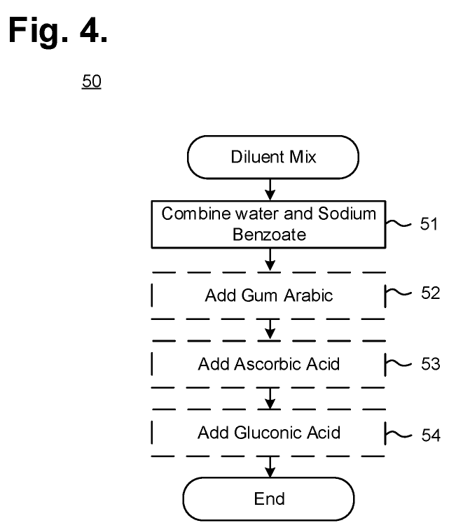

In a further embodiment, the lubricant composition can also include a Diluent mix, in addition to the THC and CBD emulsions. FIG. 4 is a flow diagram showing, by way of example, a method 50 for preparing a diluent mix. The diluent can be prepared using two or more of the components, as provided below.

TABLE 4
Diluent Mix

| | Range | Preferred Range |
|---|---|---|
| Diluent Mix | (percentage by weight) | (percentage by weight) |
| Water | 20-99% | 95-99% |
| Sodium Benzoate | 0.001-1% | 0.075-0.125% |
| Gum Arabic | 0-40% | 1-2% |
| Ascorbic Acid | 0-1% | 0.75-0.125% |
| Gluconic Acid | 0-5% | 0.5-0.75% |

The diluent mix can be prepared by adding water to a mixing vessel and adding Sodium Benzoate while mixing (step 51). Mixing is continued, for approximately 5 minutes at a speed between 2,000 to 10,000 rpm, until the Sodium Benzoate is completely dissolved. In one embodiment, the Sodium Benzoate can be mixed at 5,000 rpm. The mixing speed is increased to approximately 8,000 rpm and optionally, Gum Arabic is slowly added (step 52) to avoid lump formation. The water, Sodium Benzoate, and Gum Arabic is mixed for approximately 10 minutes after the last lump is dissolved; however, other mixing times are possible. Once dissolved, Ascorbic Acid can be optionally added (step 53) slowly, under agitation, and mixed for approximately 5 minutes. Optionally, Gluconic Acid can also be slowly added (step 54) and mixed for around five minutes. Other components in addition to or in lieu of the components described above can be used, such as those described with respect to the THC or CBD emulsions.

The diluent mix can be used to adjust the pH of the lubricant, as well as a consistency of the lubricant. FIG. 5 is a flow diagram showing, by way of example, a method 60 for preparing a lubricant based on the THC emulsion of FIG. 1, the CBD emulsion of FIG. 2, and the diluent mix of FIG. 4. The diluent lubricant can include two or more of the components, as provided below.

TABLE 5
Lubricant Composition 2

| | Range | Preferred Range |
|---|---|---|
| Lubricant Composition 2 | (percentage by weight) | (percentage by weight) |
| THC Master Mix | 10-99% | 20-30% |
| CBD Master Mix | 5-99% | 7-10% |
| Diluent Master Mix | 30-80% | 55-70% |
| Allantoin | 0-1% | 0.125-0.2% |
| Potassium Sorbate | 0-1% | 0.0075-0.015% |
| Stevia | 0-2% | 0.125-0.15% |
| Aloe Vera | 0-5% | 0.75-1.25% |
| Water | 0-10% | 1-5% |
| Cannabis Terpene | 0-10% | 0.025-0.05% |
| Hydroxyethyl Cellulose | 0-5% | 0-0.1% |
| Xanthan Gum | 0-5% | 1-2% |
| Citric Acid | 0-3% | 0% |
| Gluconic Acid | 0-5% | 0.5-0.75% |

The diluent lubricant composition can be prepared by combining (step 61) the THC emulsion, CBD emulsion, and the diluent mix, in appropriate amounts, to a large mixing container. A large hole screen, with holes approximately ¼″, is positioned on a high shear mixer and the emulsions and diluent mix are mixed at approximately 6,000 rpm. Other mixing speeds and types of mixers are possible. Optionally, Allantoin is added (step 62) and mixed, approximately 5 minutes, until dispersed. Potassium Sorbate can also be optionally added (step 63) and mixed, approximately 5 minutes, until dispersed. Further, a sweetener, such as Stevia, can be optionally added (step 64) and mixed for approximately 5 minutes, until dispersed. As well, a moisturizer, such as Aloe Vera, can be optionally added (step 65) and mixed for 5 minutes, until dispersed.

The addition (step 66) of water, such as deionized or purified water is optional and may be used when the THC and CBD concentrations require ratio adjustment. Less water is likely to be added in the diluent lubricant composition, due to the presence of the diluent mix, than in the lubricant composition described above with respect to FIG. 3. Terpenes, such as from cannabis plants, can be added (step 67) and mixed for approximately five minutes, until dispersed. In one embodiment, the terpenes can include cannabis terpenes, such as Dutch Treat, or terpenes derived from non-cannabis sources. A cellulosic, such as Hydroxyethyl Cellulose, can be optionally added (step 68) and mix at an increased speed of approximately 9,000 to 10,000 rpm, while gently stirring with a spatula to ensure all lumps of the cellulosic are dispersed. Mixing of the cellulosic with the rest of the mixture described above can occur for approximately 10 minutes. Other mixing times and types of cellulosics, such as Carboxymethyl Cellulose, are possible.

A thickening agent, such as Xanthan Gum, can also be added (step 69) to the mixture at the increased speed of approximately 3,000 to 10,000 rpm, while gently stirring with the spatula to ensure all lumps are dispersed. Also, Citric Acid can be optionally added (step 70) to the mixture, and Gluconic Acid, which generally acts as an acidity regulator, is added (step 71). Once all the desired components have been added, the mixture is mixed (step 72) for approximately 10 minutes and then packaged (step 73). The lubricant can be packed in jars, canisters, or spray or pump bottles.

Components, other than those described above, are also possible for use in the diluent lubricant composition. For example, in lieu of or in addition to Allantoin, Aloe Vera, palm oil, vegetable oil, or glycerin can be used, as well as other healing or soothing components. Additionally, in lieu of or in addition to Stevia, sweeteners, such as Sucralose, Aspartame, cyclamates, Saccharin, Aceslfame Potassium, and Neotame can be used. In lieu of or in addition to Xanthan Gum, other food grade water soluble thickeners are possible, such as hydrocolloids, minerals such as fumed silica, and starches, as well as other thickeners. Other components that can be used include Sodium Benzoate, such as a replacement or in addition to the Potassium Sorbate. Finally, other terpenes are possible.

The different lubricants as described above can be applied as a primer, rather than a traditional lubricant. Generally, 2 to 5 pumps of the lubricant can be applied to an area of an individual's body, including erogenous zones, such as the genitals, or to a device, such as a sex toy, at the beginning of sexual activity or at least 20 minutes prior to intercourse or masturbation, rather than a traditional lubricant, which is generally applied immediately prior to sexual activity. Each pump of the lubricant can include about 0.23 grams of the lubricant composition. However, other amounts of lubricant per pump, numbers of pumps, or total amount of lubricant can be used based on an individual on which the lubricant is applied. Due to the wait time, the user can also apply a different lubricant to ensure sufficient moisture is present. The wearer should experience enhanced sensation immediately and orgasmic potential upon orgasm.

The lubricant compositions as outlined in the preferred amounts in Tables 3 and 5 were provided to 38 individuals for application and use. The individuals ranged in age from 20 to 60, and included male, female, and transgender individuals. The test conditions were based on standard clinical techniques and no harm was caused to any individual. Almost 75% of the individuals experienced some effect from the lubricant, including a tingling sensation on the applied areas, as well as an increased intensity of all sensations. Additionally, many individuals had enhanced orgasms, including longer and stronger orgasms, and a quick climax. Some individuals also reported less pain on penetration during sexual intercourse or masturbation. Upon removal, the lubricant can be easily removable from the skin, clothing, and bedsheets.

While the invention has been particularly shown and described as referenced to the embodiments thereof, those skilled in the art will understand that the foregoing and other changes in form and detail may be made therein without departing from the spirit and scope of the invention. For example, other components and percentages by weight are possible for different variations of the lubricant composition that remain in scope of the current application and are hereby incorporated.

What is claimed is:

1. A lubricant composition, comprising:
THC emulsion;
CBD emulsion;
water;
terpenes; and
a thickening agent,
wherein the THC emulsion, CBD emulsion, water, cannabis terpenes, and thickening agent are combined as a lubricant.

2. A lubricant composition according to claim 1, wherein the lubricant is one of a cream and lotion that is formulated for application on an individual or a device used by the individual.

3. A lubricant composition according to claim 1, wherein the THC emulsion comprises THC and one or more of water, Sodium Benzoate, Gum Arabic, Ascorbic Acid, Citric Acid, Gluconic Acid, Mid Chain Triglycerides, and Sucrose Acetate Isobutyrate.

4. A lubricant composition according to claim 1, wherein the CBD emulsion comprises CBD and one or more of water, Sodium Benzoate, Gum Arabic, Ascorbic Acid, Citric Acid, Gluconic Acid, Mid Chain Triglycerides, and Sucrose Acetate Isobutyrate.

5. A lubricant composition according to claim 1, further comprising one or more of:
Allantoin;
Potassium Sorbate;
a sweetener substance;
Aloe Vera;
a moisturizer;
Hydroxyethyl Cellulose;
a cellulosic;
Citric Acid; and
Gluconic Acid.

6. A lubricant composition according to claim 5, further comprising a diluent mix comprising two or more of water, Sodium Benzoate, gum Arabic, Ascorbic Acid, and Gluconic Acid.

7. A lubricant composition according to claim 1, further comprising:
a diluent mix comprising one or more of water, Sodium Benzoate, gum Arabic, Ascorbic Acid, and Gluconic Acid.

8. A lubricant composition according to claim 1, wherein the terpenes comprise one or more of cannabis terpenes and terpenes derived from non-cannabis sources and further wherein the thickening agent comprises one or more of hydroxyethyl cellulose, carboxyethyl cellulose, and a cellulostic.

9. A lubricant composition according to claim 1, wherein the lubricant is applied to at least one of one or more erogenous zones on a body of an individual and a device to be used on the body.

10. A lubricant composition according to claim 1, wherein two to five pumps of the lubricant is applied.

11. A method for preparing a lubricant composition, comprising:
combining a THC emulsion with a CBD emulsion;
mixing the combined THC emulsion and CBD emulsion;
adding water to the mixed THC emulsion and CBD emulsion;
adding cannabis terpenes to the water, THC emulsion, and CBD emulsion, as a lubricant; and
adding a thickening agent to thicken the lubricant.

12. A method according to claim 11, wherein the lubricant is one of a cream and lotion that is formulated for application on an individual or a device used by the individual.

13. A method according to claim 11, wherein the THC emulsion comprises THC and one or more of water, Sodium Benzoate, Gum Arabic, Ascorbic Acid, Citric Acid, Gluconic Acid, Mid Chain Triglycerides, and Sucrose Acetate Isobutyrate.

14. A method according to claim 11, wherein the CBD emulsion comprises CBD and one or more of water, Sodium Benzoate, Gum Arabic, Ascorbic Acid, Citric Acid, Gluconic Acid, Mid Chain Triglycerides, and Sucrose Acetate Isobutyrate.

15. A method according to claim 11, further comprising:
adding to the lubricant one or more of Allantoin, Potassium Sorbate, a sweetener substance, Aloe Vera, moisturizer, Hydroxyethyl Cellulose, Xanthan Gum, a cellulosic, Citric Acid, and Gluconic Acid.

16. A method according to claim 15, further comprising:
adding to the lubricant a diluent mix comprising two or more of water, Sodium Benzoate, gum Arabic, Ascorbic Acid, and Gluconic Acid.

17. A method according to claim 11, further comprising:
adding to the lubricant a diluent mix comprising one or more of water, Sodium Benzoate, Gum Arabic, Ascorbic Acid, and Gluconic Acid.

18. A method according to claim 11, wherein the terpenes comprise one or more of cannabis terpenes and terpenes derived from non-cannabis sources and further wherein the thickening agent comprises one or more of Hydroxyethyl Cellulose, Carboxyethyl Cellulose, Xanthan Gum, and a cellulosic.

19. A method according to claim 11, wherein the lubricant is applied to at least one of one or more erogenous zones on a body of an individual and a device to be used on the body.

20. A method according to claim 11, wherein two to five pumps of the lubricant is applied.
